SEQUENCE LISTING (6 sequences)

SEQ ID NO: 1 - DNA, 21 bp, Homo sapiens
  misc_RNA (1)..(19)
  misc_feature (20)..(21): DNA overhang
ggccuucaguaaagguugatt

SEQ ID NO: 2 - DNA, 21 bp, Homo sapiens
  misc_RNA (1)..(19)
  misc_feature (20)..(21): DNA overhang
ucaaccuuuacugaaggcctg

SEQ ID NO: 3 - DNA, 21 bp, Homo sapiens
  misc_RNA (1)..(19)
  misc_feature (20)..(21): DNA overhang
guauagacaugauugacaatt

SEQ ID NO: 4 - DNA, 21 bp, Homo sapiens
  misc_RNA (1)..(19)
  misc_feature (20)..(21): DNA overhang
uugucaaucaugucuauacct

SEQ ID NO: 5 - DNA, 21 bp, Homo sapiens
  misc_RNA (1)..(19)
  misc_feature (20)..(21): DNA overhang
ggugucugaagaaugagaatt

SEQ ID NO: 6 - DNA, 21 bp, Homo sapiens
  misc_RNA (1)..(19)
  misc_feature (20)..(21): DNA overhang
uucucauucuucagacacctt

(19) United States
(12) Patent Application Publication
(10) Pub. No.: US 2019/0203211 A1
(43) Pub. Date: Jul. 4, 2019

(54) TREATMENT AND PREVENTION OF VIRAL INFECTION

(71) Applicant: The University of Birmingham, Birmingham (GB)

(72) Inventors: Xiaodong Zhuang, Oxford, Oxfordshire (GB); Jane Alison McKeating, Oxford, Oxfordshire (GB)

(73) Assignee: The University of Birmingham, Birmingham (GB)

(21) Appl. No.: 16/330,604

(22) PCT Filed: Sep. 5, 2017
(86) PCT No.: PCT/GB2017/052575

§ 371 Date: Mar. 5, 2019

(30) Foreign Application Priority Data
Sep. 5, 2016 (GB) 1615035.1

(51) Int. Cl. C12N 15/113; A61P 31/12; A61K 31/381; A61K 31/4025; A61K 31/4436; A61P 31/18; A61P 31/14

(52) U.S. Cl. CPC C12N 15/1138; A61P 31/12; A61K 31/381; C12N 2310/14; A61K 31/4436; A61P 31/18; A61P 31/14; A61K 31/4025

(57) ABSTRACT

An agent for use in a method of treating and/or preventing a viral infection, and compositions comprising said agent, are described. The agent modulates the expression of one or more circadian clock genes, or the activity of one or more circadian clock gene products. The agent may comprise or consist of an agonist of REV-ERBα.

The present invention relates to an agent for use in a method of treating and/or preventing a viral infection, and compositions comprising said agent. More particularly, the invention relates to the treatment and/or prevention of viral infection through the use of an agent that modulates the expression of one or more circadian clock genes.

Currently there are limited therapeutic choices for treating many viral infections, with many drugs showing evidence for the selection of resistant viruses. Identifying and targeting host pathways that are essential to the virus life cycle provides the potential for more efficacious therapies that provide higher barriers to the development of resistance and exhibit broad activity against a wide range of viruses. Hence, there is a global drive to identify agents that target host pathways and inhibit viral replication.

The circadian rhythm—24 hour cycling—orchestrates physiology and prepares the body to respond to environmental signals. The mammalian circadian clock is driven by a ‘core’ transcriptional feedback loop of transcriptional activators and repressors (FIG. 1). These include BMAL1 and CLOCK, which form a heterodimer which activates the transcription of REV-ERBα and ROR. In turn, the repressor REV-ERBα inhibits the activity of the CLOCK-BMAL1 complex. The CLOCK-BMAL1 complex also initiates the transcription of the clock ‘period’ genes PER1, PER2 and PER3 and the two cryptochrome genes CRY1 and CRY2. PER-CRY heterodimers inhibit their own transcription by inhibiting the activity of the CLOCK-BMAL1 complex.

A recent advance in the field of chronobiology is the realization that REV-ERBα regulates cellular metabolism and immunity. The identification of REV-ERBα natural ligands has spurred the development of synthetic ligands and opened up the possibility of targeting REV-ERBα to treat diseases including diabetes, atherosclerosis, autoimmunity and cancer.

The present invention has been devised with these issues in mind.

According to a first aspect of the invention there is provided an agent that modulates the expression of one or more circadian clock genes, or activity of one or more circadian clock gene products, for use in a method of treating or preventing a viral infection.

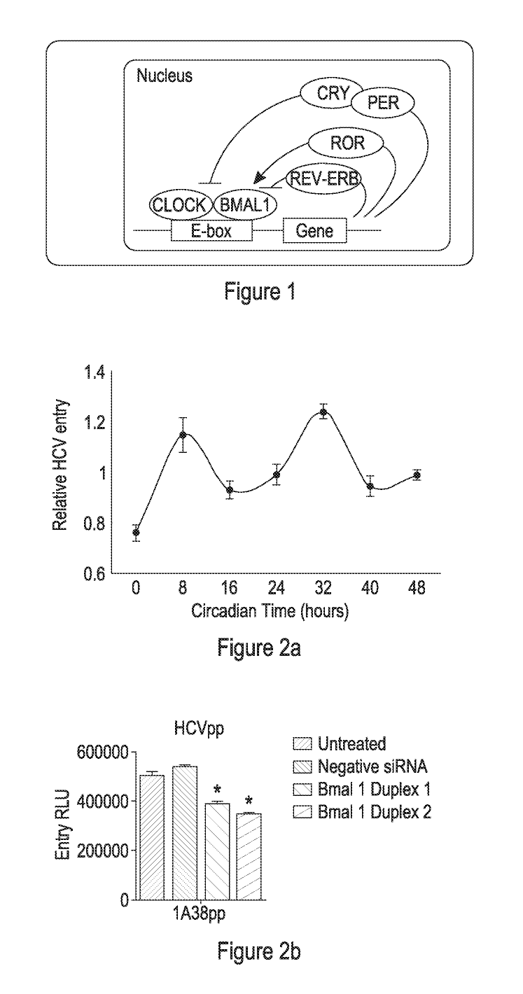

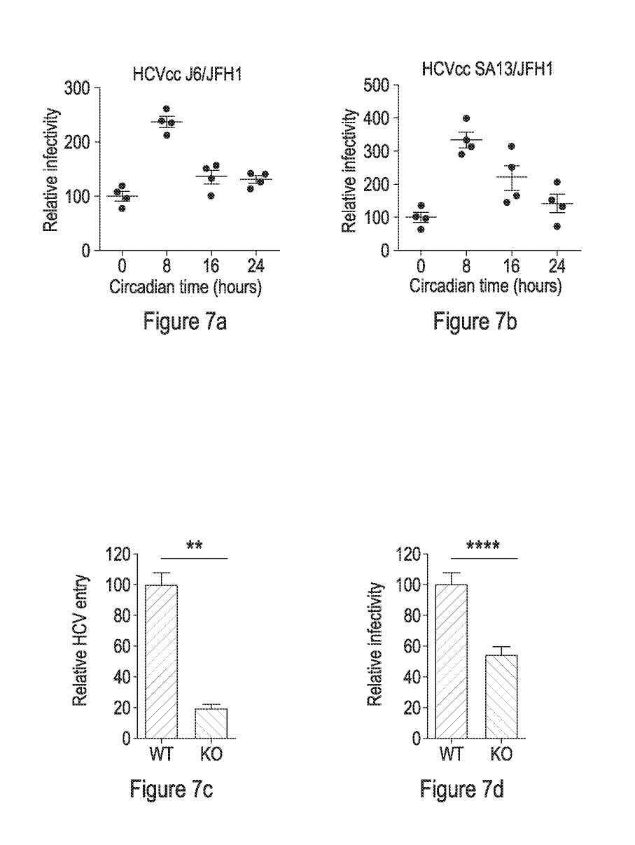

The present inventors have surprisingly found that virus infection may be circadian regulated (FIG. 2a, FIG. 7a). Prior to the present invention, the role of circadian pathways in regulating viral infection has not been studied, providing a rare opportunity for the discovery of novel anti-viral agents.

In some embodiments the agent modulates the expression of one or more circadian clock genes, or activity of one or more circadian clock gene products selected from the group consisting of BMAL1, BMAL2, CRY1, CRY2, PERI, PER2, PER3, REV-ERBα, REV-ERBβ, RORα, RORβ and CLOCK.

As used herein, “BMAL1”, “REV-ERBα” etc. will be understood as referring to the gene or the protein encoded by the gene, as appropriate.

By “modulates the expression of”, as used herein, it will be understood that the agent increases or decreases gene expression relative to normal levels (i.e. the level in the absence of the agent). It will be appreciated that whether expression is increased or decreased will depend on the nature of the agent (e.g. agonist vs. antagonist), and whether the target is an activator or a repressor in the circadian transcriptional feedback loop. It will be further appreciated that expression of a given gene may be modulated directly or indirectly. For example, the agent may be a nucleic acid that specifically binds to mRNA, thereby causing direct repression of expression of the gene into a protein. In another example, the agent may be a small molecule which indirectly causes gene expression to be decreased through activation of a transcriptional repressor, or by affecting post-translational modifications.

By “modulates the activity of”, as used herein, it will be understood that the agent increases or decreases activity of the gene expression product e.g. protein, relative to normal activity levels (i.e. the level in the absence of the agent). It will be appreciated that whether activity is increased or decreased will depend on the nature of the agent (e.g. agonist vs. antagonist), and whether the target is an activator or a repressor in the circadian transcriptional feedback loop. Gene product activity may be modulated, for example, by post-translational or post-transcriptional modification of an expressed protein, such as by altered methylation phosphorylation, histone acetylation, glycosylation and the like.

In some embodiments, the agent directly or indirectly increases the expression or activity of the one or more circadian clock genes by at least 20%, at least 30%, at least 40%, at least 50%, at least 70%, at least 80%, at least 90% or at least 100%. In some embodiments, gene expression/activity is increased by no more than 200%, no more than 150%, no more than 120%, no more than 95%, no more than 75% or no more than 60%.

In some embodiments, the agent directly or indirectly decreases the expression or activity of the one or more circadian clock genes by at least 20%, at least 30%, at least 40%, at least 50%, at least 70%, at least 80%, at least 90%, at least 95% or substantially 100%. In some embodiments, gene expression/activity is decreased by no more than 99%, no more than 85%, no more than 75%, no more than 60% or no more than 50%.

Changes in the level of gene expression can be detected, for example, by determining mRNA levels. The effect of an agent on gene expression can be determined by comparing the mRNA level in a cell that has been treated with said agent to a cell that has not been treated with the agent. Relative or absolute mRNA levels may be determined using standard techniques known to those skilled in the art, for example qPCR. Changes in protein activity can similarly be detected by determining the level of mRNA produced by the transcription of downstream genes, or detecting protein levels and/or whether or not post-translational modification of the protein has been altered.

Agents which are capable of modulating the expression of genes, or the activity of proteins, involved in the circadian feedback loop can be identified using functional assays. Such assays may conveniently enable high throughput screening of potential modulator agents. A transcription based assay can be derived by selecting transcriptional regulatory sequences (e.g. promoters) from genes involved in the circadian feedback loop, and operatively linking such promoters to a reporter gene in an expression construct. The effect of different agents can then be detected by monitoring expression of the reporter gene in host cells transfected with the expression construct. One such assay is a luminescent reporter assay in which a circadian promoter is operatively linked to a reporter gene. Commonly used reporter genes include luciferase, beta-galactosidase, alkaline phosphatase and CAT (chloramphenicol acetyl transferase). The use of a luciferase reporter assay to monitor the effect of gene knock down and pharmacologically active compounds on the circadian pathway is described by Ramanathan et al., Journal of Visualized Experiments, 2012 (67), e4234.

It may be necessary or appropriate to further test candidate compounds in vivo, such as taught in Regulation of circadian behaviour and metabolism by synthetic REV-ERB agonists, Nature, 2012

The agent may comprise or consist of a peptide, a protein, an enzyme, an antibody, a nucleic acid (e.g. a siNA or a plasmid), or a small molecule. In some embodiments the agent is a naturally-occurring or a synthetic ligand of a protein involved in the circadian feedback loop. The term “ligand” as used herein is understood to mean a substance that binds to a biological macromolecule, such as a protein or nucleic acid, for example, to form a complex. Formation of the complex may induce a change in the function or activity of the biological macromolecule. A ligand may be an agonist or an antagonist. As used herein, the term “agonist” refers to a molecule which binds to a biological macromolecule and activates a biological response. An “antagonist” is a molecule which binds to a biological macromolecule and inhibits a biological response.

As used herein, a “small molecule” is a chemical compound having a molecular weight of no more than 2000 daltons (Da). In some embodiments, the small molecule has a molecular weight of no more than 1000, such as no more than 700 or no more than 500 Da. The small molecule may be an organic compound. The small molecule may bind to a component of the circadian feedback loop and modulate its activity and/or interactions with other proteins or nucleic acids, for example.

In some embodiments the agent comprises or consists of an antisense molecule (e.g. an antisense DNA or RNA molecule or a chemical analogue) or a ribozyme molecule. Ribozymes and antisense molecules may be used to inhibit the transcription of a gene encoding a protein involved in the circadian transcriptional feedback loop, or translation of the mRNA of that gene. Antisense molecules are oligonucleotides that bind in a sequence-specific manner to nucleic acids, such as DNA or RNA. When bound to mRNA that has a complementary sequence, antisense RNA prevents translation of the mRNA. Triplex molecules refer to single antisense DNA strands that bind duplex DNA forming a colinear triplex molecule, thereby preventing transcription. Particularly useful antisense nucleotides and triplex molecules are ones that are complementary to or bind the sense strand of DNA (or mRNA) that encodes a protein involved in the circadian transcriptional feedback loop.

In some embodiments, the agent comprises or consists of a short interfering nucleic acid (siNA). A siNA molecule may comprise a siDNA molecule or a siRNA molecule. In some embodiments, the agent comprises or consists of miRNA (microRNA), siRNA (small interfering RNA) or shRNA (short hairpin RNA). In some embodiments, the agent is a siRNA. Oligonucleotides including siNAs can be prepared by solid phase chemical synthesis using standard techniques.

In some embodiments, the agent comprises or consists of a CRISPR knockout or activation product. CRISPR knockout products, such as CRISPR/Cas9 knock-out plasmids, are commercially available and enable the identification and cleavage of a gene of interest, thereby eliminating production of the gene product. CRISPR activation products activate endogenous gene transcription.

In some embodiments, the agent is a peptide, a protein, an enzyme, an antibody or an antibody fragment (such as a Fab or F(ab′)₂ fragment, an scFV antibody, a diabody or any other functional antigen-binding fragment). Proteins and peptides may be generated using a variety of methods, including purification of naturally-occurring proteins, recombinant protein production and de novo chemical synthesis. Methods for generating antibodies are well-known to those skilled in the art.

In the circadian feedback loop, the positive elements include members of the basic helix-loop-helix (bHLH)-PAS transcription factor family, CLOCK and BMAL1. The circadian clock relies on the genes CLOCK and BMAL1 to drive expression and regulate biological functions which are under circadian control. The CLOCK and BMAL1 proteins heterodimerize and initiate transcription of target genes, including PER and CRY genes, by binding to an E-box promoter element. PER-CRY heterodimers regulate their own transcription through negative feedback by acting on the BMAL1-CLOCK complex. The BMAL1-CLOCK heterodimer also activates transcription of the retinoic acid-related orphan nuclear receptors REV-ERBα and RORα. In turn, ROR proteins (α, β and γ) activate transcription of BMAL1 while REV-ERB proteins (α and β) repress transcription of BMAL1. It will therefore be appreciated that activation of REV-ERBα will result in repression (i.e. decreased expression) of BMAL1, while activation of RORα will result in increased expression of BMAL1. Similarly, regulation of PER and/or CRY may be expected to have an effect on virus infectivity. For example the small molecule KL001 (see Science. 2012 Aug. 31; 337(6098):1094-7. doi: 10.1126/science.1223710. Epub 2012 Jul. 12) is known to activate and/or stabilize Cry. Whilst KL001 is thought to stabilize Cry protein without affecting bmal or Clock RNA, it does inhibit Bna1 promoter activity and so may be expected in accordance with the present invention to inhibit virus infectivity.

In some embodiments, the agent directly or indirectly reduces the activity or expression of BMAL1, CLOCK, or the BMAL1-CLOCK heterodimer. The agent may reduce expression by inhibiting transcription of the BMAL1 or CLOCK gene into mRNA. In some embodiments, the agent reduces the production of an active protein by inhibiting the translation of mRNA. In some embodiments, the agent inhibits post-translational modification of the translated protein.

In some embodiments, the agent is an antagonist of the BMAL1 or CLOCK protein, or an antagonist of the BMAL1-CLOCK heterodimer. An example of a BMAL1 modulator is described in “Identification of a novel circadian dock modulator controlling BMAL1 expression through a ROR/REV-ERB-response element-dependent mechanism”, 2016, Biochemical and Biophysical Research Communications.

In some embodiments, the activity or expression of BMAL1, CLOCK or the BMAL1-CLOCK heterodimer is reduced by at least 30%, at least 40%, at least 50%, at least 70%, at least 80%, at least 90% or at least 95%. In some embodiments, the activity or expression of BMAL1, CLOCK or the BMAL1-CLOCK heterodimer is reduced by no more than 99%, no more than 90%, no more than 85%, no more than 75% or no more than 60%.

In some embodiments the agent directly or indirectly decreases the expression of BMAL1. In some embodiments the agent is a nucleic acid, such as a siRNA. Examples of siRNA molecules (each comprising a two-base DNA overhang) that decrease expression of BMAL1 include:

(SEQ ID NO 1)
5′-3′ GGCCUUCAGUAAAGGUUGAtt
(SEQ ID NO 2)
5′-3′ UCAACCUUUACUGAAGGCCtg;
and
(SEQ ID NO 3)
5′-3′ GUAUAGACAUGAUUGACAAtt
(SEQ ID NO 4)
5′-3′ UUGUCAAUCAUGUCUAUACct

REV-ERB proteins are members of the nuclear receptor family of intracellular transcription activators. There are two forms of the protein, α and β, which are encoded by the genes NR1D1 and NR1D2 respectively. References herein to the REV-ERBα or REV-ERBβ gene, or expression of REV-ERBα or REV-ERBβ, will be understood as referring to the gene NR1D1 or NR1D2, or expression thereof, as appropriate.

In some embodiments, the agent increases the activity or expression of REV-ERBα. The agent may be an agonist of the REV-ERBα protein. In some embodiments, the agonist is a natural ligand of REV-ERBα. Heme is a known natural ligand of REV-ERBα and REV-ERBβ.

In some embodiment, the agonist is a synthetic ligand of REVERBα. Synthetic agonists of REV-ERBa include: 1,1-Dimethylethyl N-[(4-chlorophenyl)methyl]-N-[(5-nitro-2-thienyl)methyl])glycinate; N-Benzyl- N-(4-chlorobenzyl)-I-(5-nitrothiophen-2-yl)methanamine; N-Benzyl-N-(3,4-dichlorobenzyl)-1-(5-nitrothiophen-2-yl)methanamine; 2-((4-chlorobenzyl)((5-nitrothiophen-2-yl)methyl)amino)-N,N-dimethylacetamide; SR9009; GSK4112 and SR9011. Additional rev-erb agonists are derivatives of 6-subsituted triazolopyridines as described in WO2013/045519 to which the skilled reader is directed and the entire contents of which are hereby incorporated by way of reference.

In some embodiments, the agonist of REV-ERBa is selected from GSK4112 (also known as SR6452), SR9009, SR9011 and GSK2667.

In some embodiments, the agent is not a Rev-erb-modulating agent (REMA). A REMA affects the activity of REV-ERB (REV-ERBα and/or REV-ERBβ) by altering expression, by increasing or decreasing activity, by altering cellular localization or by other means.

In some embodiments, the agent is not an agonist of REV-ERBα.

In some embodiments, the agent is not SR6452, SR9009, SR901 or GSK2667.

In some embodiments, the activity or expression of REV-ERBα is increased by at least 30%, at least 40%, at least 50%, at least 70%, at least 80%, at least 90% or at least 100%. In some embodiments, the activity or expression of REV-ERBα is increased by no more than 200%, no more than 150% or no more than 120%.

The agent may exert an anti-viral effect by inhibiting viral entry into cells. Thus, in some embodiments the agent protects cells from viral infection. Additionally or alternatively, the agent may inhibit replication of the virus. Thus, the agent may be capable of reducing the viral load of infected cells. In some embodiments, the agent has a dual anti-viral action through inhibiting both the entry and replication processes of the virus life cycle. The effect of any agent on viral entry and/or inhibition can be determined using the methods described herein.

Thus, in some embodiments, the agent inhibits virus cell entry and/or replication. In this manner, viral entry and/or replication may be reduced by at least 30%, at least 40%, at least 50%, at least 70%, at least 80%, at least 90% or at least 95%. In some embodiments, viral entry and/or replication may be reduced by no more than 99%, no more than 90%, no more than 85%, no more than 75% or no more than 60%.

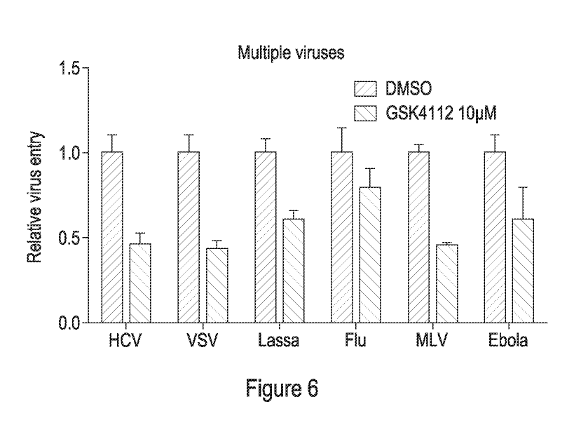

The lentiviral pseudoparticle system is a well-established model for studying viral glycoprotein-receptor dependent entry and can be applied to studying a wide range of heterologous viral glycoproteins as shown, for example, in FIG. 6. Pseudoparticles are generated by co-transfecting human embryonal kidney cells (HEK), for example, with plasmids encoding an envelope deficient disabled HIV-luciferase genome and viral glycoprotein under test. Secreted particles are collected posttransfection (e.g. after 48 h) and used to infect naïve target cells.

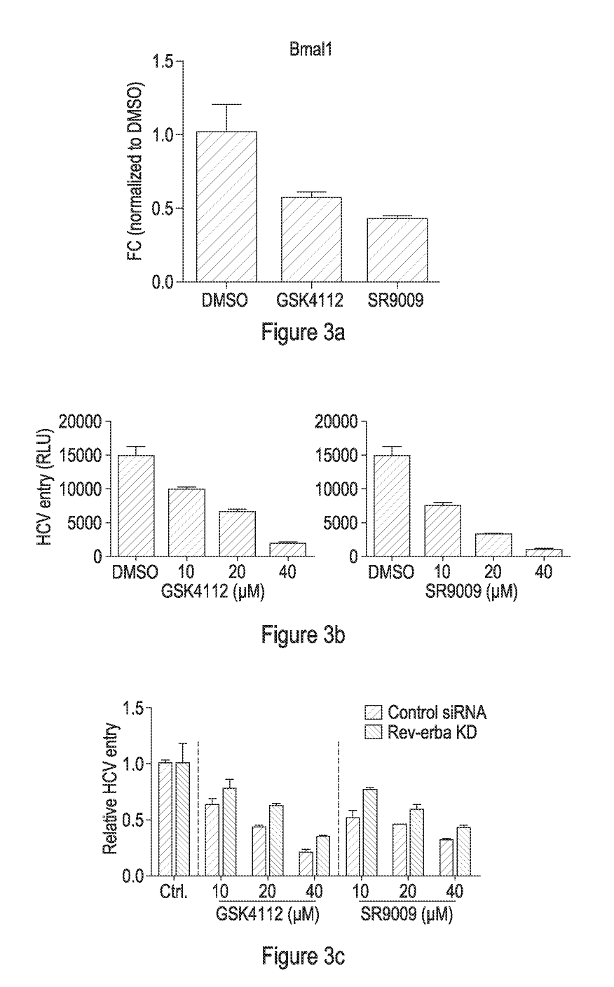

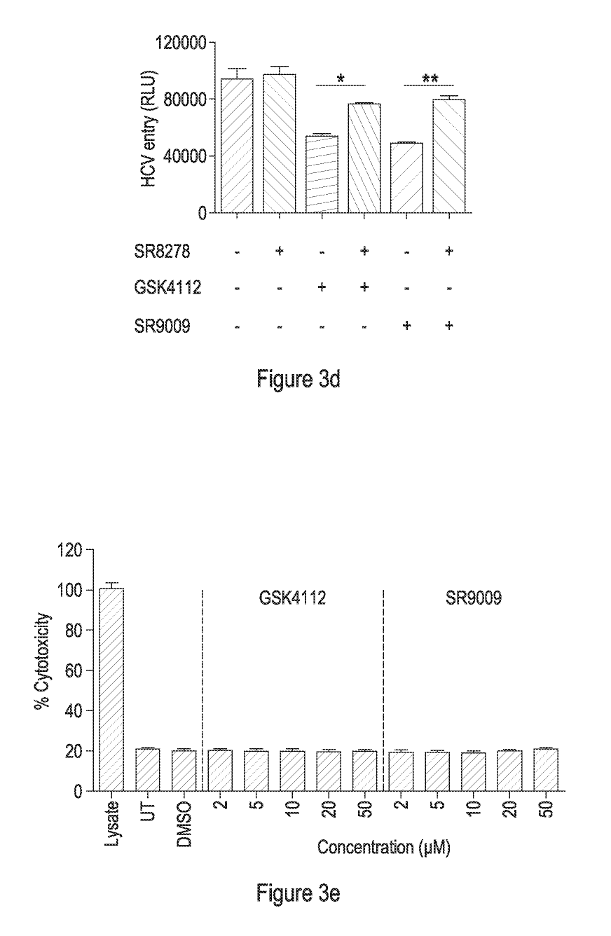

In brief, with reference to FIG. 3B as an example, human hepatoma cells Huh-7 were treated with GSK4112 or SR9009 at a range of concentration for 16 hours. The drug was removed and cells infected with HCVpp for 1 hour. Unbound virus was removed by washing and the cells cultured for 24 h before lysing and quantifying luciferase activity, as detailed in Hsu 2003 PNAS 100: 7271-6.

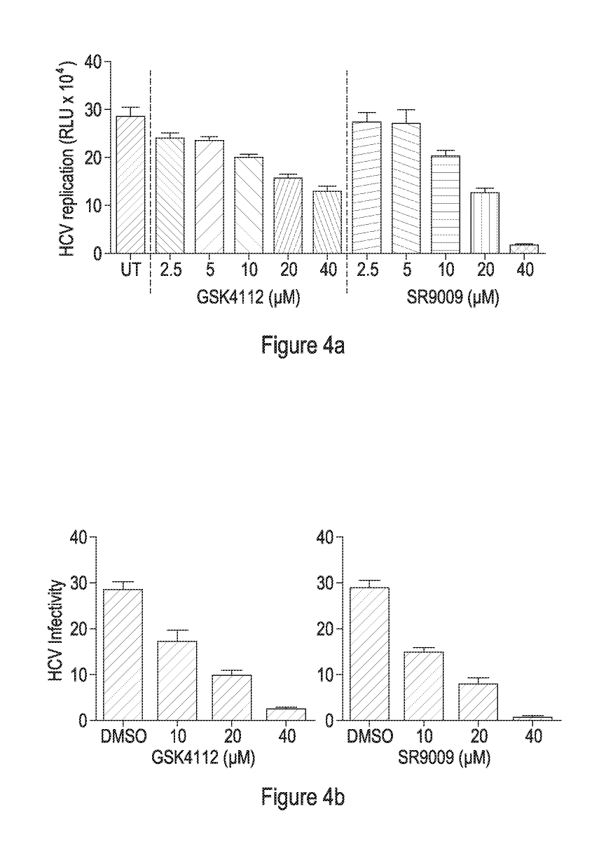

An example of how to test for agents which are capable of inhibiting viral entry/replication is described with reference to FIG. 4. HCV was generated by electroporating HCV genomic RNA into Huh-7.5 cells as detailed in Lindenbach 2005 Science 309: 623-6. Huh-7 cells were treated with GSK4112 or SR9009 for 16 h and inoculated with HCV for 1 h and the cells cultured for 24 h before fixing and staining for virus NSSA expression. Virus infection was enumerated by counting NSSA expressing cells (FIG. 4a).

To assess the ability of REV-ERB activators to limit HCV RNA replication, HCV infected Huh-7 cells (verified by NS5A staining) were treated with GSK4112 or SR9009 at a range of doses. 16 hours later, HCV infection levels were quantified by reverse transcriptase polymerase chain measurement of viral RNA (see FIG. 4b).

It will therefore be understood that the agent of the present invention may directly affect viral infection (i.e. the ability of the virus to cause disease), rather than merely treating or preventing symptoms of the infection, or other conditions which are related to or caused by the viral infection. Without being bound by theory, it is believed that REV-ERB activators modulate HCV entry by regulating tight junction claudin-1 or occludin expression, cellular factors that are essential for HCV infection (Meredith 2012 Rev Med Virol 22:182-93). Since the tight junction protein occludin regulates epithelial polarity and contributes to barrier formation that limits pathogen infection—we suggest this as a potential mechanism for REV-ERB agonists to limit the entry of a wide range of viruses. miR122 is circadian regulated and is known to be important in HCV RNA transcription and translation—providing a mechanism for REV-ERB agonists to regulate HCV and HBV replication.

By “inhibiting viral entry” and “inhibiting replication”, it will be understood that viral entry/replication may be partially or completely inhibited.

The viral infection may be caused by pathogenic viruses, such as hepatitis B, hepatitis C, vesicular stomatitis virus, Lassa virus, influenza, murine leukemia virus, ebola, HIV, Zika virus or any other suitable pathogenic animal or human virus

In some embodiments, the viral infection is not caused by viral hepatitis.

In some embodiments, the viral infection is not caused by hepatitis C or hepatitis B.

In some embodiments, the agent that modulates the expression of one or more circadian clock genes is used in combination with a further therapeutic agent.

The agent and the further therapeutic agent may be administered concomitantly, sequentially or alternately.

In some embodiments, the further therapeutic agent is an anti-viral agent.

Suitable anti-viral agents (such as agents which inhibit viral entry, replication, viral integration (anti-integrase), viral assembly and viral export and secretion) may be, for example, Adamantane antivirals, Interferons, Non-nucleoside reverse transcriptase inhibitors (NNRTIs), Chemokine receptor antagonists, Neuraminidase inhibitors, Non-structural protein 5A (NS5A) inhibitors, anti-retrovirals, Nucleoside reverse transcriptase inhibitors (NRTIs)), DNA polymerase inhibitors, Protease inhibitors, Nucleoside analogues, direct-acting antivirals (DAAs), or any combination thereof.

In some embodiments, the anti-viral agent is a DAA. There are four different classes of DAAs (NS3/4A Protease Inhibitors, Nucleoside and Nucleotide NS5B Polymerase Inhibitors (e.g. Sofosbuvir), NS5A inhibitors (e.g. Daclatasvir) and Non-nucleoside NS5B polymerase inhibitors. DAAs are mainly used in the treatment of HCV. The most suitable class for treatment will depend on the genotype of the HCV.

In some embodiments, the anti-viral agent is not an anti-HCV or an anti-HBV agent.

According to a second aspect of the invention there is provided a method of treating or preventing a viral infection, the method comprising administration of a therapeutically effective amount of an agent according to the first aspect of the invention to a subject in need thereof.

As used herein, “treating” or “treatment” refers to reducing or alleviating symptoms associated with the viral infection, inhibiting further progression or worsening of the symptoms, reducing the viral load and/or eliminating the infection. In some embodiments, the viral infection is treated by inhibiting viral entry into cells and/or inhibiting viral replication within cells. As used herein, “preventing” or “prevention” refers to protecting a subject from infection, or lessening the effect, duration or symptoms of an infection.

Also provided is the use of an agent according to the first aspect of the invention in the manufacture of a medicament for the treatment or prevention of a viral infection.

As used herein, a “therapeutically effective amount” is an amount of the agent according to the first aspect of the invention which, when administered to a subject, is sufficient to eliminate, reduce or prevent viral infection. A therapeutically effective amount may also be an amount at which there are no toxic or detrimental effects, or a level at which any toxic or detrimental effects are outweighed by the therapeutic benefits.

In some embodiments, the subject is a mammal. In some embodiments, the subject is human.

Non-human subjects to which the invention is applicable include pets, domestic animals, wildlife and livestock, including dogs, cats, cattle, horses, sheep, goats, deer and rodents.

The subject may have been diagnosed as suffering from a viral infection. The subject may be suspected of having a viral infection, and/or may be displaying symptoms of a viral infection. In some embodiments, the subject is identified as being at risk of developing a viral infection.

In some embodiments, the subject is not suffering from, suspected of suffering from or exhibiting symptoms of hepatic fibrosis and/or related pathologies such as cirrhosis and hepatocellular carcinoma.

In some embodiments, the subject is not suffering from or suspected of suffering from chronic viral hepatitis.

In some embodiments, the subject is not diagnosed as suffering from, suspected of suffering from or at risk of developing hepatitis B or hepatitis C.

Administration of the agent may be by any suitable route, including but not limited to, injection (including intravenous (bolus or infusion), intra-arterial, intraperitoneal, subcutaneous (bolus or infusion), intraventricular, intramuscular, or subarachnoidal), oral ingestion, inhalation, topical, via a mucosa (such as the oral, nasal or rectal mucosa), by delivery in the form of a spray, tablet, transdermal patch, subcutaneous implant or in the form of a suppository. The mode of administration may depend on the virus being treated. For example, some respiratory viruses may conveniently be treated by administering the agent directly to the respiratory system, for example by inhalation using an inhaler or nebulizer.

According to a third aspect of the invention there is provided a composition comprising a therapeutically effective amount of at least one agent according to the first aspect of the invention. The composition may be described as an anti-viral composition.

In some embodiments the composition is a vaccine composition.

The composition or vaccine composition may further comprise a pharmaceutically acceptable carrier. A “pharmaceutically acceptable carrier” as referred to herein is any physiological vehicle known to those of ordinary skill in the art useful in formulating pharmaceutical compositions. The agent may be mixed with, or dissolved, suspended or dispersed in the carrier.

The composition may be in the form of a capsule, tablet, liquid, ointment, cream, gel, hydrogel, aerosol, spray, micelle, transdermal patch, liposome or any other suitable form that may be administered to a mammal suffering from, or at risk of developing, a viral infection.

In embodiments wherein the agent is a peptide or protein, a nucleic acid sequence encoding the peptide or protein may be provided in a suitable vector, for example a plasmid, a cosmid or a viral vector. Thus, also provided is a vector (i.e. a construct), comprising a nucleic acid sequence which encodes the protein or peptide. The nucleic acid sequence is preferably operably linked to a suitable promoter. The invention further relates to a composition comprising the vector.

Agents which are nucleic acids, such as siRNAs or miRNAs, may be modified (e.g. via chemical modification of the nucleic acid backbone), or delivered in suitable delivery system which protects the nucleic acids from degradation and/or immune system recognition. Examples of suitable delivery systems include nanoparticles, lipid particles, polymer-mediated delivery systems, lipid-based nanovectors and exosomes.

In some embodiments, a dose of between 0.1 μg/kg of body weight and 1 g/kg of body weight of an agent according to the first aspect of the invention may be administered for the treatment or prevention of viral infection, depending upon the specific agent used.

The agent may be administered as a single dose or as multiple doses. Multiple doses may be administered in a single day (e.g. 2, 3 or 4 doses at intervals of e.g. 3, 6 or 8 hours). The agent may be administered on a regular basis (e.g. daily, every other day, or weekly) over a period of days, weeks or months, as appropriate.

It will be appreciated that optimal doses to be administered can be determined by those skilled in the art, and will vary depending on the particular agent in use, the strength of the preparation, the mode of administration, the advancement or severity of the infection, and the type of virus. Additional factors depending on the particular subject being treated will result in a need to adjust dosages, including subject age, weight, gender, diet, and time of administration. Known procedures, such as those conventionally employed by the pharmaceutical industry (e.g. in vivo experimentation, clinical trials, etc.), may be used to establish specific formulations for use according to the invention and precise therapeutic dosage regimes.

In some embodiments, the composition additionally comprises a further therapeutic agent. The further therapeutic agent may be an anti-viral agent.

All of the features described herein (including any accompanying claims, abstract and drawings) may be combined with any of the above aspects in any combination, unless otherwise indicated.

Embodiments of the invention will now be described by way of example and with reference to the accompanying figures, in which:

FIG. 1 is a diagram of the core circadian feedback loop;

FIG. 2a is a plot showing the change in HCV entry into cells over time;

FIG. 2b is a graph showing the effect of Bmal1 knockdown on HCV entry;

FIG. 3a is a graph showing the effect of Rev-erbα activators on Bmal1 mRNA levels;

FIG. 3b is a graph showing the effect of Rev-erbα activators on HCV infectivity;

FIG. 3c is a graph showing the effect of Rev-erbα knockdown on HCV entry in cells treated with Rev-erbα agonists;

FIG. 3d is a graph showing the effect of a Rev-erbα antagonist on HCV entry in cells treated with Rev-erbα agonists;

FIG. 3e is a graph showing cytotoxic activity in cells treated with Rev-erbα agonists;

FIG. 4a is a graph showing the effect of Rev-erbα agonists on HCV replication;

FIG. 4b is a graph showing the effect of Rev-erbα agonists on HCV infectivity;

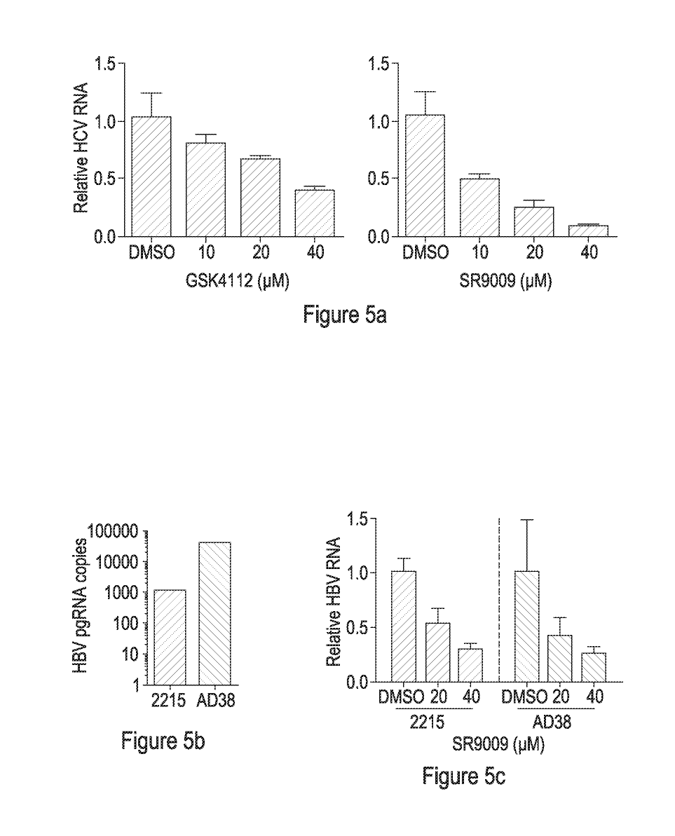

FIG. 5a shows graphs showing the effect of Rev-erbα agonists on HCV mRNA levels;

FIG. 5b is a graph showing the HBV pre-genomic RNA (pgRNA) burden in two cell lines;

FIG. 5c shows graphs showing the effect of Rev-erbα agonists on the HBV RNA levels in the cell lines of FIG. 5b;

FIG. 6 is a graph showing the effect of Rev-erbα agonists on cell entry by pseudoparticles expressing a range of viral glycoproteins;

FIGS. 7a and 7b are graphs indicating that HCV infection shows a circadian pattern;

FIGS. 7c and 7d are graphs showing that BMAL1 regulates HCV entry and infection;

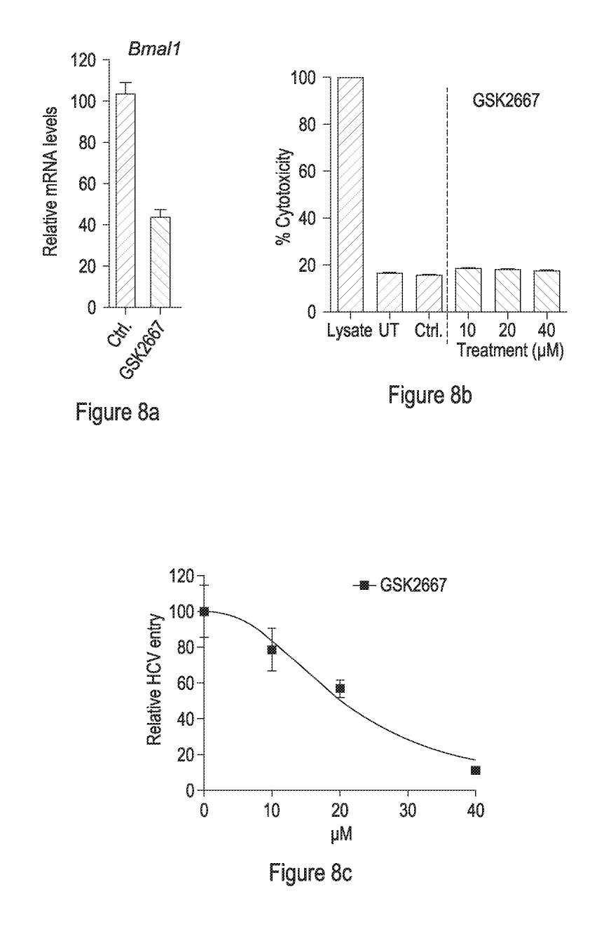

FIG. 8a is a graph showing the effect of a Rev-erb agonist on the Bmal1 mRNA level;

FIG. 8b is a plot showing the effect of a Rev-erb agonist on cell viability;

FIG. 8c is a graph showing the effect of a Rev-erb agonist on HCV entry;

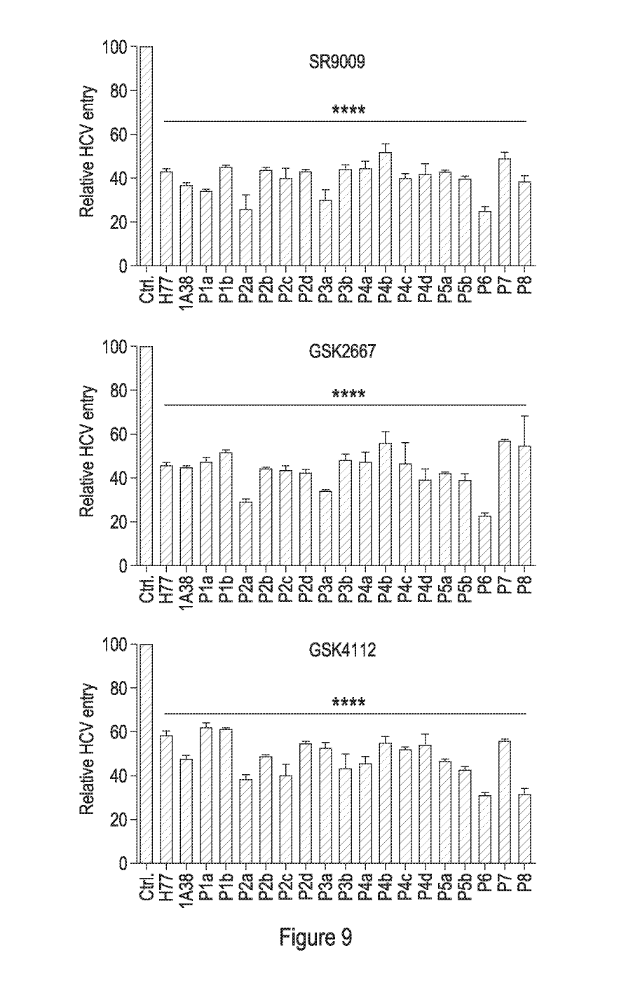

FIG. 9 shows graphs showing the effect of Rev-erb agonists on entry by HCV pseudoparticles expressing patient derived glycoproteins;

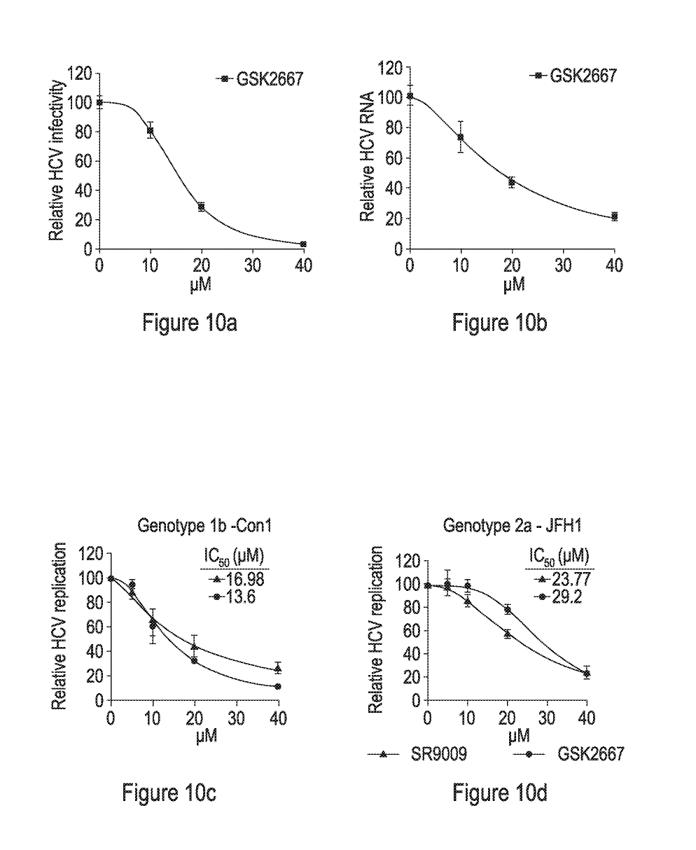

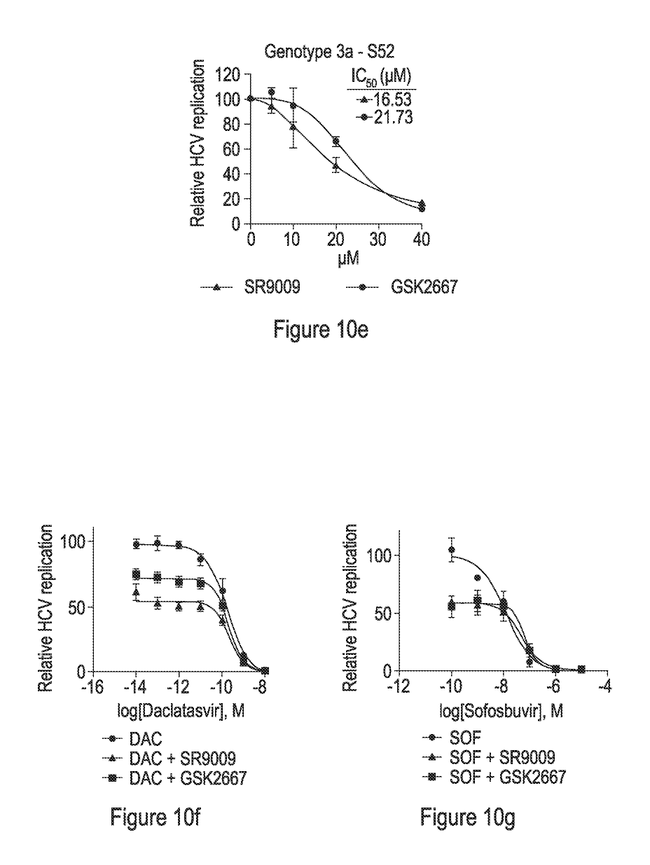

FIG. 10a is a graph showing the relative HCV infectivity in Huh-7 cells treated with a Rev-erb agonist;

FIG. 10b is a graph showing HCV RNA levels in cells treated with a Rev-erb agonist;

FIGS. 10c-10e are graphs showing the effect of Rev-erb agonists on HCV replication for genotypes 1 (FIG. 10c), 2 (FIG. 10d) and 3 (FIG. 10f);

FIG. 10f is a graph showing the additive effect of Rev-erb agonists and Daclatasvir on HCV replication;

FIG. 10g is a graph showing the additive effect of Rev-erb agonists and Sofosbuvir on HCV replication;

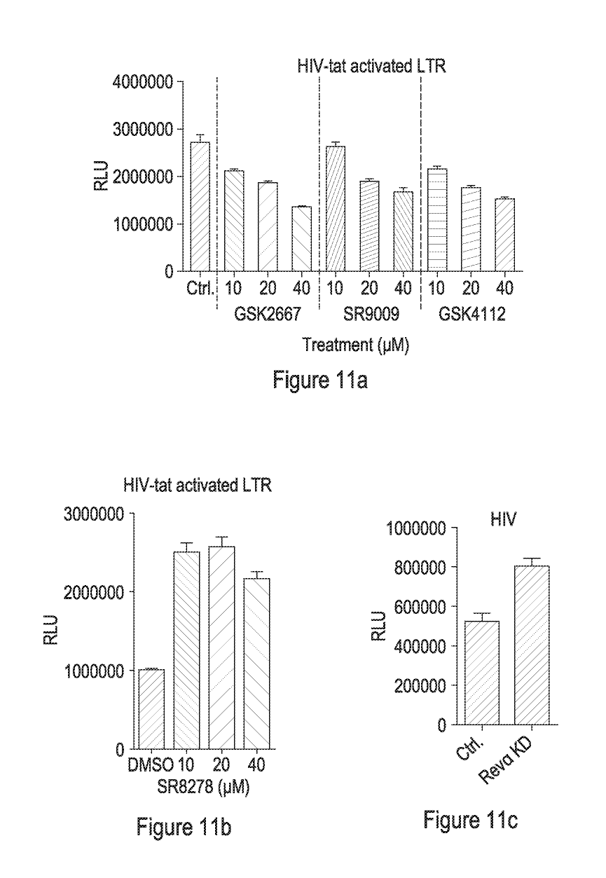

FIG. 11a is a graph showing the effect of Rev-erb agonists on HIV infection in TZM-bl cells;

FIG. 11b is a graph showing of a Rev-erb antagonist on HIV infection in TZM-bl cells;

FIG. 11c is a graph showing the effect of Rev-erba silencing on HIV infection in TZM-bl cells; and

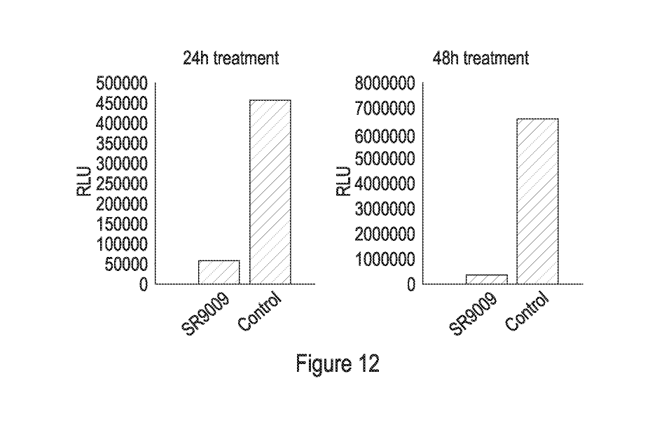

FIG. 12 is a graph showing the effect of a Rev-erb agonist on Zika virus infection in Huh-7 cells.

### EXAMPLE 1

### Introduction

With reference to FIG. 1, the core circadian gene oscillator comprises an interlocking loop of transcriptional activators and repressors that cycle every 24 hours. The loop comprises the heterodimeric activators CLOCK and BMAL1, which dimerize in the cytoplasm to form a complex. A major regulatory loop is induced when CLOCK:BMAL1 heterodimers translocate into the nucleus and activate the transcription of rev-erba and rora, two retinoic acid-related orphan nuclear receptors. REV-ERBa and RORa subsequently compete to bind retinoic acid-related orphan receptor response elements (ROREs) present in Bmal1 promoter. Through the subsequent binding of ROREs, members of ROR and REV-ERB are able to modulate Bmal1 level. While RORs activate transcription of Bmal1, REV-ERBs repress Bmal1 transcription, thus the circadian rhythm of Bmal1 is both positively and negatively regulated by RORs and REV-ERBs. The CLOCK-BMAL1 complex also initiates the transcription of the clock ‘period’ genes PER1, PER2 and PER3 and the two cryptochrome genes CRY1 and CRY2 by binding to the E-box present in their promoters. PER-CRY heterodimers inhibit their own transcription by inhibiting the activity of the CLOCK-BMAL1 complex.

### Materials and Methods

### Cells and Reagents

All cells were maintained in Dulbecco's modified Eagle medium (DMEM) supplemented with 10% fetal bovine serum (FBS), 1% nonessential amino acids/1% penicillin/streptomycin

(Invitrogen, UK). REV-ERBα agonists GSK4112 and SR9009 and antagonist SR8278 were purchased from Sigma, UK.

### Generation of Viral Pseudoparticles and Infectivity Measurement

Luciferase reporter pseudoparticles expressing a panel of viral envelope glycoproteins: hepatitis C virus—HCV; vesicular stomatitis virus—VSV; Lassa virus—Lassa; Influenza—Flu, Murine Leukemia virus—MLC and Ebola virus were generated as reported by Hsu, M., et al. (Hepatitis C virus glycoproteins mediate pH-dependent cell entry of pseudotyped retroviral particles. Proc Natl Acad Sci U S A, 2003. 100(12): p. 7271-6). Virus-containing medium was added to target cells and incubated for 24 hours. Cells were lysed and luciferase activity was measured. Infectivity is expressed as relative light units (RLU).

### Serum Shock Synchronization

Huh-7 cells were synchronized with a treatment of 50% fetal bovine serum in the standard medium for 1 hour. After the serum shock, the old medium was replaced with fresh DMEM containing 3% FBS. Cells at circadian times (CTs) across 48 hour with 8-hour intervals were then challenged with HCV virus and infectivity evaluated 24 hours later.

### Treatment with REV-ERBα Modulators

Huh-7 cells were treated with either REV-ERBa agonist GSK4112 or SR9009 for 16 hour at a range of doses. Medium containing the drug was then removed following viral inoculation and infectivity assessed 24 hours later. To evaluate the efficacy of these drugs in viral replication, chronic infected HCV or HBV cells were treated with REV-ERBa activators at a range of concentrations and viral load determined by real-time qPCR 24 hours later.

### Knockdown by siRNA Silencing

Bmal1 and rev-erba siRNA duplexes were purchased from Life technologies, UK. The Bma1 sequences are identified above. The rev-erba SiRNA sequences (each comprising a two-base

DNA overhang) are:

(SEQ ID NO 5)
5′-3′ GGUGUCUGAAGAAUGAGAAtt
(SEQ ID NO 6)
5′-3′ UUCUCAUUCUUCAGACACCtt

The transfection mix was prepared using DharmaFECT 4 (GE Dharmacon, UK) following manufacture instructions. 48-hour or 72-hour post siRNA transfection at 25 nM, cells were treated with REV-ERBa modulators following viral infection as described above.

### Results and Discussion

Synchronized hepatocytes were challenged with HCV at circadian times and infection was assessed 24 hours later as described above. The results show that hepatitis C virus (HCV) entry into host targets cells is circadian regulated (FIG. 2a). BMAL1 silencing by siRNA was found to reduce HCV entry into the hepatocytes (FIG. 2b).

Hepatocytes were treated with REV-ERBα activators following evaluation of rev-erba mRNA levels by qPCR. It was shown that pharmacological activation of REV-ERBα (BMAL1 repressor) using the commercially available ligands GSK4112 or SR9009 decreased Bmal1 mRNA levels (FIG. 3a). It was also shown that these ligands protect naïve cells from HCV infection in a dose-dependent manner (FIG. 3b). This anti-viral activity was rescued by siRNA silencing rev-erba or treating with a REV-ERBα antagonist (SR8278) (FIG. 3c and d), indicating a specific mode of action through REV-ERBα activation. Neither GSK4112 nor SR9009 showed any detectable cytotoxicity at the concentrations shown to have anti-viral activity (FIG. 3e).

Replication of the HCV genome in a synthetic sub-genomic replicon line was inhibited by both REV-ERBα agonists GSK4112 or SR9009in a dose-dependent manner (FIG. 4a). Naïve cells treated with the REV-ERBα agonists were also found to be protected from full HCV virus challenge in a dose-dependent manner (FIG. 4b)

The ability of REV-ERBα activators to reduce viral burden of chronic HCV infected cells was then tested. Chronic HCV infected cells were treated with an increasing dose of GSK4112 or SR9009 for 24 hours and viral genomic RNA was quantified by qPCR. A dose-dependent reduction in viral load was observed with both drugs (FIG. 5a). An identical assay was performed in two chronic HBV infected cell lines—2215 and AD38. HBV pre-genomic RNA (pgRNA) burden was assessed, with AD38 exhibiting a heavier viral burden (10-fold) compared with 2215 (FIG. 5b). Treating these cells with SR9009 for 48 hours significantly reduced HBV RNA levels (FIG. 5c).

It was then investigated whether the REV-ERBα activation has a wider anti-viral spectrum against the entry of other human viruses. Naïve cells were treated with GSK4112 at a suboptimal dose and the effect on entry of pseudoparticles expressing a range of viral encoded glycoproteins was determined (hepatitis C virus—HCV; vesicular stomatitis virus—VSV; Lassa virus—Lassa; Influenza—Flu; Murine Leukemia Virus—MLC and Ebola virus—Ebola). It was found that even with a sub-optimal dosage, the inhibitory effect on entry was replicated across multiple viruses including lassa and ebola (FIG. 6).

These studies show that activation of REV-ERBα, and consequent BMAL1 repression, can both protect naïve cells from viral infection and reduce viral replication in chronically infected cells. This provides a promising anti-viral therapy by targeting the circadian pathway.

### EXAMPLE 2: HCV Infection is Circadian Regulated

Authentic HCVcc particles (strains J6/FH and SA13/JFH) were used and demonstrated a significant increase in HCV NSSA expressing cells when inoculated at CTB, supporting a model where HCV infection is circadian regulated. Plasmids encoding HCV SA13/JFH and J6/JFH were used to generate RNA and electroporated into Huh-7 cells. Infected cells were fixed with ice old methanol, stained for viral antigen expression with mAb specific for NSSA (9E10) and isotype-matched Alexa-488 conjugated IgG. Viral antigen expressing cells were enumerated using a fluorescent microscope. HCV RNA levels were assessed by quantitative reverse-transcription polymerase chain reaction (qRT-PCR).

With reference to FIG. 7, synchronized Huh-7 cells were inoculated with HCVcc J6/JFH-1 (FIG. 7a) or SA13/JFH-1 (FIG. 7b) at defined CTs and the frequency of infected cells quantified 24h later and the data expressed relative to CT0. The data indicates that HCV infection shows a circadian pattern.

Bmal1 knockout Huh-7 clones were generated with transfection of a pool of three BMAL1 CRISPR/Cas9 KO Plasmids (Santa Cruz Biotechnology) following FACs sorting and clonal expansion. Parental or Bmal1 KO Huh-7 cell lysates were assessed for BMAL1 and housekeeping GAPDH by Western blotting. Parental or Bmal1 KO Huh-7 cells were inoculated for 1 h with HCVpp (1A38) (FIG. 7c) or HCVcc SA13/JFH-1 (FIG. 7d) and infection assessed after 24 h. A significant reduction in HCVpp entry and HCVcc infection in the Bmal1 KO cells was observed.

### EXAMPLE 3: Effect of REV-ERB Agonist GSK2667

Huh-7 cells were treated with the REV-ERB agonist GSK2667 for 24 h and Bmal1 and GAPDH mRNA levels quantified by RT-qPCR. It was found that GSK2667 reduces Bmal1 transcripts (FIG. 8a) and protein.

The effect of REV-ERB agonist GSK2667 on Huh-7 viability was tested. Huh-7 cells were treated with GSK2667 at increasing doses for 48 h and cytotoxicity assessed by LDH assay. GSK2667 did not show any detectable cytotoxicity at the concentrations tested (FIG. 8b).

Huh-7 cells were treated with an increasing dose of REV-ERB agonist GSK2667 for 24 h, inoculated with HCVpp (1A38) and infection assessed 24 h later. As shown in FIG. 8c, REV-ERB agonist GSK2667 inhibited HCV entry.

### EXAMPLE 4: REV-ERB Agonists Inhibit HCVPP Expressing Patient Derived Glycoproteins

To evaluate the activity of REV-ERB agonists against a wider spectrum of HCV strains, we used lentiviral pseudotypes expressing primary envelope glycoproteins cloned from patients with acute HCV infection [1]. All three ligands showed broad activity against a panel of HCVpp strains. Luciferase reporter pseudoparticles expressing HCV envelope glycoproteins (HCVpp), or no-glycoprotein controls, were generated in 293T cells using a plasmid encoding a HIV provirus expressing luciferase and viral envelope glycoproteins from lab strains H77 and 1A38 and HCV patient derived clones [2]. Huh-7 cells were treated with the REV-ERB agonists GSK2667, SR9009 or GSK4112 (20 μM) for 24 h and infected with HCVpp expressing patient derived envelope glycoproteins and infectivity assessed 24 h later. It was found that the compounds are able to inhibit HCV entry not only of lab strains but also a wide range of patient derived HCV (FIG. 9).

### EXAMPLE 5: REV-ERB Agonists Inhibit HCV Replication

Huh-7 cells were treated with an increasing concentration of REV-ERB agonist GSK2667 for 24 h, inoculated with HCVcc SA13/JFH-1 and infectivity measured after 24 h. As shown in FIG. 10a, GSK2667 inhibited HCV infection, the relative HCV infectivity decreasing as the concentration of GSK2667 increased.

HCVcc SA13/JFH-1 infected Huh-7 cells were also treated with increasing concentrations of GSK2667 for 24 h and viral RNA levels measured after 24 h. As shown in FIG. 10b, treatment with GSK2667 is able to cure HCV-infected cells.

Direct acting antiviral agents (DAAs) are revolutionising how we treat chronic hepatitis C with >90% cure rates in subjects infected with genotype 1 and 2 viruses. In contrast, genotype 3 HCV is more refractory to DAAs and the underlying mechanism for this resistance is likely to be multi-factorial. This is supported by co-treating Huh-7 cells with REV-ERB agonists (20 μM) and increasing concentrations of direct acting antiviral agents Daclatasvir and Sofosbuvir, targeting NS5A and NS5B, respectively, to inhibit HCV genotype 3a S52 replication. We found that combining REV-ERB agonists with Daclatasvir or Sofosbuvir showed additive effects to inhibit HCV genotype 3 replication.

The plasmids encoding the HCV subgenomic replicons were generated as previously reported [3]. Specifically, the L-GDD con1 (genotype 1b), JFH1-luc (genotype 2a) and S52-ΔN (genotype 3a) were linearized with XbaI (New England Biolabs, NEB), treated with Mung Bean nuclease (NEB) and purified linearised templates used to generate in vitro transcribed RNAs [4]. 2 μg of RNA was electroporated into 4×10⁶ cells, which were allowed to recover for 48 h before treating with REV-ERB ligands.

Huh-7.5-SEC14L2 cells transiently supporting HCV sub-genomic replicons encoding a luciferase (Luc) reporter representing genotypes 1-3 were treated with increasing concentrations of REV-ERB agonists SR9009 or GSK2667 and replication assessed 24 h later. The IC50 of each REV-ERB agonist to inhibit HCV RNA replication by 50% (IC50) was calculated against individual HCV genotype. It was observed that the antiviral activity of the REV-ERB agonists is pan-genomic (FIGS. 10c-e).

FIGS. 10f and 10g show the additive effects of REV-ERB agonists and DAAs. Huh-7.5-SEC14L2 cells were electroporated with HCV genotype 3a S52-Luc replicon RNA and subsequently treated with DAAs targeting NS5A (Daclatasvir—DAC) or viral polymerase NS5b (Sofosbuvir—SOF) for 48 h in the presence or absence of REV-ERB agonists SR9009 and GSK2667. Luciferase activity was measured 24 h later.

### EXAMPLE 6: REV-ERB Agonists Inhibit Hiv Infection in TZM-bl Cells

The TZM-bl cell line is highly sensitive to infection with diverse isolates of HIV-1 or HIV protein TAT treatment. It enables simple and quantitative analysis of HIV using luciferase as a reporter. The cell line was generated by introducing separate integrated copies of the luciferase and β-galactosidase genes under control of the HIV-1 promoter.

TZM-bl cells were infected with HIV virus (NL4.3) in the presence of an increasing dose of REV-ERB agonists GSK2667, SR9009 or GSK4112 or antagonist SR8278. 24 h later, the HIV promoter activity was measured in luciferase assay. The REV-ERB agonists were found to inhibit HIV infection (FIG. 11a).

TZM-bl cells were infected with HIV in the presence of increasing concentration of REV-ERB antagonist SR8278 for 24 h and infectivity measured after 24 h. It was observed that the REV-ERB antagonist promotes HIV infection (FIG. 11b).

48 h post-siRNA knockdown of Rev-erbα, TZM-bl cells were infected with HIV for 24 h and infectivity measured after 24 h. siRNA knockdown of Rev-erbα in the TZM-bl cells led to increased HIV promoter activity, confirming that the effect of REV-ERB modulators was specific (FIG. 11c).

### EXAMPLE 7: REV-ERB Agonists Inhibit Zika Infection

Huh-7 cells were infected with Zika virus in the present of REV-ERB agonists SR9009 and infectivity measured after 24 h and 48 h. The Zika virus was generated based on the Asian lineage sequence and encodes a duplicated capsid protein surrounding the nanoluciferase gene/2A/ ubiquitin sequence. As shown in FIG. 12, REV-ERB agonists inhibit Zika infection.

### REFERENCES

1. Bailey, J. R., et al., Naturally selected hepatitis C virus polymorphisms confer broad neutralizing antibody resistance. J Clin Invest, 2015. 125(1): p. 437-47.
2. Fafi-Kremer, S., et al., Viral entry and escape from antibody-mediated neutralization influence hepatitis C virus reinfection in liver transplantation. J Exp Med, 2010. 207(9): p. 2019-31.
3. Witteveldt, J., M. Martin-Gans, and P. Simmonds, Enhancement of the Replication of Hepatitis C Virus Replicons of Genotypes 1 to 4 by Manipulation of CpG and UpA Dinucleotide Frequencies and Use of Cell Lines Expressing SECL14L2 for Antiviral Resistance Testing. Antimicrob Agents Chemother, 2016. 60(5): p. 2981-92.
4. Magri, A., et al., 17,beta-estradiol inhibits hepatitis C virus mainly by interference with the release phase of its life cycle. Liver Int, 2016.

1. A method of treating or preventing a viral infection, the method comprising administering a therapeutically effective amount of an agent that modulates the expression of one or more circadian clock genes, or activity of one or more circadian clock gene products, to a subject in need thereof.

2. The method according to claim 1, wherein the circadian clock genes are selected from the group consisting of BMAL1, Bmal2, Cry1, Cry2, Pert Per2, Per3, REV-ERBα, Rev-erbβ, ROR, and CLOCK.

3. The method according to claim 1, wherein the agent inhibits the activity or expression of Bmal1, CLOCK, or the BMAL1-CLOCK heterodimer.

4. The method according to claim 3, wherein the agent comprises an antagonist of BMAL1, CLOCK, or the BMAL1-CLOCK heterodimer.

5. The method according to claim 3, wherein the agent directly or indirectly represses the transcription or translation of BMAL1.

6. The method according to claim 5, wherein the agent comprises a nucleic acid.

7. The method according to claim 6, wherein the nucleic acid is a siRNA.

8. The method according to claim 5, wherein the agent increases the expression or activity of REV-ERBα.

9. The method according to claim 8, wherein the agent comprises an agonist of REV-ERBα.

10. The method according to claim 9, wherein the agonist comprises a synthetic or naturally-occurring ligand of REV-ERBα.

11. The method according to claim 10, wherein the agonist of REV-ERBα comprises or consists of GSK4112, SR9009, SR9011, or GSK2667.

12. The method according to claim 1, wherein the viral infection is caused by a pathogenic virus.

13. An anti-viral composition comprising a therapeutically effective amount of an agent that modulates the expression of one or more circadian clock genes, or activity of one or more circadian clock gene products, wherein said therapeutically effective amount is sufficient to reduce, treat, cure, or prevent a viral infection.

14. The composition of claim 13, wherein said composition is a vaccine composition and further comprises a pharmaceutically acceptable carrier.

15. The method of claim 12, wherein the pathogenic virus is selected from hepatitis B, hepatitis C, vesicular stomatitis virus, Lassa virus, influenza, murine leukemia virus, HIV, Zika virus, and ebola.
